SEQUENCE LISTING (43 sequences)

SEQ ID NO: 1 - protein, 560 aa, Artificial Sequence
  CRM197
MSRKLFASILIGALLGIGAPPSAHAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 792 aa, Artificial Sequence
  HIN47-HID
MTLPSFVSEQNSLAPMLEKVQPAVVTLSVEGKAKVDSRSPFLDDIPEEFKFFFGDRFAEQFGGRGESKRNFRGLGSGVIINASKGYVLTNNAVIDEADKITVQLQDGREFKAKLVGKDELSDIALVQLEKPSNLTEIKFADSDKLRVGDFTVAIGNPFGLGQTVTSGIVSALGRSTGSDSGTYENYIQTDAAVNRGNSGGALVNLNGELIGINTAIISPSGGNAGIAFAIPSNQASNLVQQILEFGQVRRGLLGIKGGELNADLAKAFNVSAQQGAFVSEVLPKSAAEKAGLKAGDIITAMNGQKISSFAEIRAKIATTGAGKEISLTYLRDGKSHDVKMKLQADDSSQLSSKTELPALDGATLKDYDAKGVKGIEITKIQPNSLAAQRGLKSGDIIIGINRQMIENIRELNKVLETEPSAVALNILRGDSNFYLLVQGGSGGSMDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 3 - protein, 791 aa, Artificial Sequence
  HID-HIN47
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIKGGSGGSTLPSFVSEQNSLAPMLEKVQPAVVTLSVEGKAKVDSRSPFLDDIPEEFKFFFGDRFAEQFGGRGESKRNFRGLGSGVIINASKGYVLTNNAVIDEADKITVQLQDGREFKAKLVGKDELSDIALVQLEKPSNLTEIKFADSDKLRVGDFTVAIGNPFGLGQTVTSGIVSALGRSTGSDSGTYENYIQTDAAVNRGNSGGALVNLNGELIGINTAIISPSGGNAGIAFAIPSNQASNLVQQILEFGQVRRGLLGIKGGELNADLAKAFNVSAQQGAFVSEVLPKSAAEKAGLKAGDIITAMNGQKISSFAEIRAKIATTGAGKEISLTYLRDGKSHDVKMKLQADDSSQLSSKTELPALDGATLKDYDAKGVKGIEITKIQPNSLAAQRGLKSGDIIIGINRQMIENIRELNKVLETEPSAVALNILRGDSNFYLLVQ

SEQ ID NO: 4 - DNA, 39 bp, Artificial Sequence
  Primer
ccgatgtagcctagaaatttcctcataggcataggaaag

SEQ ID NO: 5 - DNA, 32 bp, Artificial Sequence
  Primer
tttctaggctacatcggttaagccgtccagaa

SEQ ID NO: 6 - DNA, 33 bp, Artificial Sequence
  Primer
gaccaaataggggaaacaggctcaagtgtatcc

SEQ ID NO: 7 - DNA, 32 bp, Artificial Sequence
  Primer
gtttcccctatttggtctcgaaattttccgtc

SEQ ID NO: 8 - DNA, 32 bp, Artificial Sequence
  Primer
aaaacctagctccttcctcagatgggaatgga

SEQ ID NO: 9 - DNA, 35 bp, Artificial Sequence
  Primer
ggaaggagctaggtttttatgcgtttcagttcatt

SEQ ID NO: 10 - DNA, 38 bp, Artificial Sequence
  Primer
cgaaaggatagtgggtaaactacaattatgattggatg

SEQ ID NO: 11 - DNA, 32 bp, Artificial Sequence
  Primer
tacccactatcctttcggatctttttcctggg

SEQ ID NO: 12 - DNA, 32 bp, Artificial Sequence
  Primer
tagagtaccgggtcagactccggcacctacga

SEQ ID NO: 13 - DNA, 37 bp, Artificial Sequence
  Primer
tctgacccggtactctagcccagagcggaaactatcc

SEQ ID NO: 14 - DNA, 32 bp, Artificial Sequence
  Primer
tgctatctagtcaccgagcggcggtaatgcgg

SEQ ID NO: 15 - DNA, 37 bp, Artificial Sequence
  Primer
tcggtgactagatagcagtattgataccgatcaattc

SEQ ID NO: 16 - DNA, 32 bp, Artificial Sequence
  Primer
ttaggcgggaattgcgttcgcaattcccagta

SEQ ID NO: 17 - DNA, 34 bp, Artificial Sequence
  Primer
acgcaattcccgcctaaccgccgctcggtgagat

SEQ ID NO: 18 - DNA, 32 bp, Artificial Sequence
  Primer
aatgcgggataggcgttcgcaattcccagtaa

SEQ ID NO: 19 - DNA, 32 bp, Artificial Sequence
  Primer
aacgcctatcccgcattaccgccgctcggtga

SEQ ID NO: 20 - DNA, 35 bp, Artificial Sequence
  Primer
gctgaaatttaggcaaaaattgctacaactggtgc

SEQ ID NO: 21 - DNA, 33 bp, Artificial Sequence
  Primer
tttgcctaaatttcagcaaacgaagagattttc

SEQ ID NO: 22 - DNA, 32 bp, Artificial Sequence
  Primer
tcgcgcatagattgctacaactggtgcaggca

SEQ ID NO: 23 - DNA, 32 bp, Artificial Sequence
  Primer
tagcaatctatgcgcgaatttcagcaaacgaa

SEQ ID NO: 24 - DNA, 39 bp, Artificial Sequence; identical to SEQ ID NO: 4
  Primer

SEQ ID NO: 25 - DNA, 32 bp, Artificial Sequence; identical to SEQ ID NO: 5
  Primer

SEQ ID NO: 26 - DNA, 33 bp, Artificial Sequence; identical to SEQ ID NO: 6
  Primer

SEQ ID NO: 27 - DNA, 32 bp, Artificial Sequence; identical to SEQ ID NO: 7
  Primer

SEQ ID NO: 28 - DNA, 32 bp, Artificial Sequence; identical to SEQ ID NO: 8
  Primer

SEQ ID NO: 29 - DNA, 35 bp, Artificial Sequence; identical to SEQ ID NO: 9
  Primer

SEQ ID NO: 30 - DNA, 38 bp, Artificial Sequence; identical to SEQ ID NO: 10
  Primer

SEQ ID NO: 31 - DNA, 32 bp, Artificial Sequence; identical to SEQ ID NO: 11
  Primer

SEQ ID NO: 32 - DNA, 32 bp, Artificial Sequence; identical to SEQ ID NO: 12
  Primer

SEQ ID NO: 33 - DNA, 37 bp, Artificial Sequence; identical to SEQ ID NO: 13
  Primer

SEQ ID NO: 34 - DNA, 32 bp, Artificial Sequence; identical to SEQ ID NO: 14
  Primer

SEQ ID NO: 35 - DNA, 37 bp, Artificial Sequence; identical to SEQ ID NO: 15
  Primer

SEQ ID NO: 36 - DNA, 32 bp, Artificial Sequence; identical to SEQ ID NO: 16
  Primer

SEQ ID NO: 37 - DNA, 34 bp, Artificial Sequence; identical to SEQ ID NO: 17
  Primer

SEQ ID NO: 38 - DNA, 32 bp, Artificial Sequence; identical to SEQ ID NO: 18
  Primer

SEQ ID NO: 39 - DNA, 32 bp, Artificial Sequence; identical to SEQ ID NO: 19
  Primer

SEQ ID NO: 40 - DNA, 35 bp, Artificial Sequence; identical to SEQ ID NO: 20
  Primer

SEQ ID NO: 41 - DNA, 33 bp, Artificial Sequence; identical to SEQ ID NO: 21
  Primer

SEQ ID NO: 42 - DNA, 32 bp, Artificial Sequence; identical to SEQ ID NO: 22
  Primer

SEQ ID NO: 43 - DNA, 32 bp, Artificial Sequence; identical to SEQ ID NO: 23
  Primer

(19) United States
(12) Patent Application Publication
(10) Pub. No.: US 2023/0111095 A1
(43) Pub. Date: Apr. 13, 2023

(54) CARRIER PROTEIN WITH SITE-DIRECTED MUTATION AND USE THEREOF IN PREPARATION OF VACCINE

(71) Applicant: Cansino Biologics Inc., Tianjin (CN)

(72) Inventors: Haomeng WANG, Tianjin (CN); Qiaoling YAN, Tianjin (CN); Shoubai CHAO, Tianjin (CN); Huhua MAO, Tianjin (CN); Tao ZHU, Tianjin (CN)

(21) Appl. No.: 17/769,607

(22) PCT Filed: Sep. 25, 2020
(86) PCT No.: PCT/CN2020/117825

§ 371 Date: Apr. 15, 2022

(30) Foreign Application Priority Data
Oct. 15, 2019 (CN) 201910978406.X

(51) Int. Cl. A61K 39/385; A61P 31/04; A61K 39/09

(52) U.S. Cl. CPC A61K 39/385; A61P 31/04; A61K 39/092; A61K 2039/6043

(57) ABSTRACT

The present invention relates to a carrier protein with site-directed mutation and use thereof in preparation of a vaccine, wherein the carrier protein is selected from fusion proteins formed by one, two or more of diphtheria toxoid, a non-toxic mutant of diphtheria toxin, a bacterial outer membrane protein and a bacterially expressed protein, wherein an amino acid at at least one site on the carrier protein is mutated into an unnatural amino acid, and the unnatural amino acid contains an azido or alkynyl terminal group. In a mutual reaction process of the carrier protein with site-directed mutation of the present invention and a polysaccharide antigen, a covalent bond is formed, and meanwhile a formed conjugate is in a bead-string state, so that the carrier protein and the polysaccharide antigen can be effectively prevented from being excessively crosslinked. Further, particle size distribution of the conjugate is significantly uniform and controllable, which provides an effective means for improving quality of a polysaccharide-protein conjugate vaccine.

### TECHNICAL FIELD

The present invention relates to the field of biological pharmacy, and in particular to a carrier protein with site-directed mutation and use thereof in preparation of a vaccine, especially a multivalent conjugate vaccine for pneumonia.

### BACKGROUND

Infection by Streptococcus pneumoniae is one of the leading causes for morbidity and mortality worldwide. Pneumonia, febrile bacteremia and meningitis are the most common manifestations of invasive pneumococcal diseases, and dissemination of bacteria in the respiratory tract can lead to middle ear infection, nasosinusitis or recurrent bronchitis.

In polysaccharide-protein conjugate vaccines prepared by linking polysaccharide to a protein carrier, chemical bonds of the polysaccharide and the protein carrier can induce an immune response against bacteria that display on their surface the polysaccharide contained in the vaccine, thereby preventing diseases. Therefore, vaccination using polysaccharides from pathogenic bacteria is a potential strategy to enhance host immunity.

Although polysaccharides are immunogenic on their own, conjugation of polysaccharide to carrier protein has been used to improve immunogenicity. The carrier protein may be either a related protein antigen that comes from a target pathogen and enhances a specific immune response against that pathogen, or a generally immunogenic protein that primarily serves as an adjuvant or a general immune response stimulant.

A traditional polysaccharide-protein conjugation technology, such as a preparation method for polysaccharide-protein conjugate vaccine disclosed in Chinese patent ZL02159032.X, is also one of the most commonly used technologies for preparing polysaccharide conjugate vaccines at present. In the described technology, adipic acid dihydrazide (ADH) is used as a linker to conjugate the polysaccharide to the protein. With respect to this conjugation mode, firstly the polysaccharide needs to be activated by cyanogen bromide, and that is, cyanogen bromide is used for acting on hydroxyl group on polysaccharide molecule under an alkaline condition to form cyanate ester, and then the cyanate ester reacts with ADH; one carbon-oxygen bond in the cyanate ester is broken, and the cyanate ester is subjected to addition reaction with an amino group at one end of the ADH, so that ester hydrazide (AH) group is introduced into polysaccharide molecule to form polysaccharide-AH derivative; the polysaccharide-AH derivative forms a stable conjugate with the carrier protein under the mediation of carbodiimide (EDAC). Such conjugation mode can reduce the steric hindrance of the conjugation of the polysaccharide to the carrier protein, reserves the epitope of the polysaccharide, and meanwhile avoids the solubility of the polysaccharide and thus reduces the side effect of the reaction of the polysaccharide and the antiserum. However, this conjugation mode has the following deficiencies: (1) the polysaccharide-AH derivative will continuously react with the polysaccharide activated by cyanogen bromide to form a self-polymer of the polysaccharide, so that the conjugation efficiency of the polysaccharide to the protein is reduced; (2) EDAC is easy to cause self-crosslinking of the carrier protein while mediating the conjugation of the ADH-derived polysaccharide to the carrier protein, thereby reducing the conjugation efficiency of the polysaccharide to the protein. Therefore, the immunogenicity and antibody persistence of the polysaccharide-protein conjugate vaccine still need to be further improved; for example, vaccination with polysaccharide conjugate vaccine needs to be performed three times to generate immune effect. These deficiencies have limited further development of polysaccharide conjugate vaccines.

Modification in biomolecular systems using the Click reaction of azido and alkynyl is a hotspot of research in recent years. Natalie W. Nairn et al. introduced an unnatural amino acid containing azido into interferon β using a methionine-deficient strain, followed by single-site PEG modification using a copper-catalyzed Click reaction (Natalie W. Narin et al., Bioconjugate Chem. 2012).

In recent years, the genetic code expansion technology develops rapidly. By using an amber stop codon as a sense codon and by introducing corresponding orthogonal tRNA and aminoacyl tRNAsynthetase, a designed unnatural amino acid can be finally introduced into a protein, so that special functions can be endowed to the protein based on the properties of the unnatural amino acid. So far, this technology has successfully and site-directly expressed several dozen kinds of unnatural amino acids in the proteins of living cells, and the involved unnatural amino acids comprise alkynyl, azido, etc. Site-directed modification of proteins can be made specifically by utilizing these special groups that do not originally exist in organisms. In order to solve such problems in the prior art as inhomogeneity in the process for conjugate vaccines and complex separation and purification process, a method capable of specifically modifying functional groups at any site is urgently needed in the field.

CN106929482A describes influenza virus with site-directed mutation, live vaccine thereof, preparation method therefor and use thereof, and the modified carrier protein Hin47-HID of the present invention has been proved to be capable of preventing non-invasive Haemophilusinfluenzae. In a specific embodiment of the present invention, provided are a mutant carrier protein Hin47-HID site-directedly introduced with a non-natural amino acid and a method for performing site-directed modification thereof. In this mutation method, the specific azido of the unnatural amino acid Lys-azido introduced at a specific site in the protein can specifically be in Click reaction with a modifier and thereby site-directly couples the non-natural amino acid to the target protein.

### SUMMARY

The present invention relates to an immunogenic composition with site-directed mutation and site-directed modification, a preparation method therefor and use thereof. For the immunogenic composition, an amber codon TAG is introduced at a specific site of a carrier protein gene, and orthogonal aminoacyl tRNAsynthetase-tRNA is used to site-directly mutate an unnatural amino acid with cross-linking property to the specific site of the carrier protein. In a mutual reaction process of the carrier protein and a polysaccharide antigen, a covalent bond is formed, and meanwhile a formed conjugate is in a bead-string state, so that the carrier protein and the polysaccharide antigen can be effectively prevented from being excessively crosslinked. Further, particle size distribution of the conjugate is significantly uniform and controllable, which provides an effective means for improving quality of a polysaccharide-protein conjugate vaccine. Based on thinking and research on the prior art, the inventor utilizes a protein translation system of tRNA (tRNA^{Pyl}) and pyrrolysine-tRNAsynthetase (tRNA^{Pyl}/PylRS) of Methanococcus archaea to allow an unnatural amino acid to be site-directly incorporated into a protein, so as to obtain a target peptide or protein with site-directed mutation, such as HID-Hin47, Hin47-HID or CRM197, and then the carrier protein with site-directed mutation, as a raw material which can be further site-directly modified, is conjugated to a polysaccharide to obtain a polysaccharide vaccine of a carrier protein with site-directed modification at a single site.

Accordingly, the present invention provides a carrier protein with site-directed mutation, wherein the carrier protein is selected from fusion proteins formed by one, two or more of diphtheria toxoid, a non-toxic mutant of diphtheria toxin, a bacterial outer membrane protein and a bacterially expressed protein, wherein an amino acid at at least one site on the carrier protein is mutated into an unnatural amino acid, and the unnatural amino acid contains an azido or alkynyl terminal group.

The carrier protein is selected from fusion proteins formed by one, two or more of diphtheria toxoid, non-toxic mutant of diphtheria toxin CRM197 (SEQ ID NO: 1), group B meningococcal outer membrane protein, recombinant Pseudomonas aeruginosa exotoxin A, Haemophilusinfluenzae lipoprotein HID and Haemophilusinfluenzae heat shock protein HIN47. Preferably, the carrier protein is selected from Haemophilusinfluenzae lipoprotein HID, fusion protein HIN47-HID and fusion protein HID-HIN47.

The unnatural amino acid is a phenylalanine derivative, a tyrosine derivative, a glutamine derivative, an alanine derivative, a cysteine derivative, a serine derivative or a lysine derivative. Preferably, the unnatural amino acid is a lysine derivative containing azido. More preferably, the unnatural amino acid is:

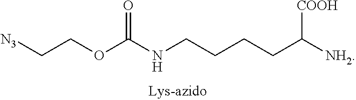 [SMILES: [N-]=[N+]=NCCOC(=O)NCCCCC(N)C(=O)O]

In one embodiment of the present invention, the carrier protein is the HIN47-HID fusion protein, and the mutation site may be one or more amino acids at any site in SEQ ID NO: 2; preferably, the mutation site is selected from position 174R, position 217I, position 223N, position 226I, position 313R, position 315K, position 522K, position 557D, position 653E, position 684Y and other sites which have small influence on activity on a sequence set forth in SEQ ID NO: 2.

In one embodiment of the present invention, the carrier protein is the HID-HIN47 protein, and the mutation site may be one or more amino acids at any site in SEQ ID NO: 3; preferably, the mutation site is selected from position 78K, position 113D, position 209E, position 240Y, position 527R, position 570I, position 576N, position 579I, position 666R, position 668K and other sites which have small influence on activity on a sequence set forth in SEQ ID NO: 3.

After mutation, the difference between the amino acid sequence of the protein with site-directed mutation and the amino acid sequence of the target protein before mutation is that the amino acid at position N on the amino acid sequence of the protein before mutation is mutated into Lys-azido, and the linking mode of the mutated amino acid is shown as the following formula:

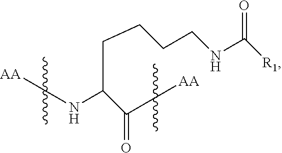

wherein, the N is a mutation site, and AA is an amino acid

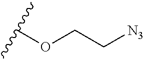 [SMILES: *OCCN=[N+]=[N-]]

located before and after the mutation site.

The present invention also provides a conjugate of a carrier protein with site-directed mutation, wherein the conjugate is prepared by the carrier protein with site-directed mutation of the present invention and a molecule containing or modified to have an alkynyl terminal group, wherein the molecule is a saccharide, a polynucleotide, an amino acid, a polypeptide or a small molecule compound containing an alkynyl terminal group, or is a modified substance obtained by modification of a saccharide, a polynucleotide, an amino acid, a polypeptide or a small molecule compound with an alkynyl terminal group.

The carrier protein with site-directed mutation of the present invention and a molecule containing or modified to have an alkynyl terminal group are subjected to a Click reaction to obtain the conjugate. The Click reaction can be a Click reaction mediated by monovalent copper and can also be a copper-free Click reaction mediated by cyclooctyne or a derivative thereof.

The saccharide, the polynucleotide, the amino acid, the polypeptide or the small molecular compound of the present invention can be a modified substance obtained by modification of a saccharide, a polynucleotide, an amino acid, a polypeptide or a small molecule compound with an alkynyl terminal group, which realizes site-directed coupling by the catalysis of monovalent copper catalyst to obtain the conjugate. Alternatively, the saccharide, the polynucleotide, the amino acid, the polypeptide or the small molecular compound is a modified substance with cyclooctyne or a derivative thereof as a modifying group, which directly realizes site-directed coupling.

Preferably, in the conjugate of the carrier protein with site-directed mutation of the present invention, the amino acid at position N on the amino acid sequence of the protein before mutation is mutated into the following structure:

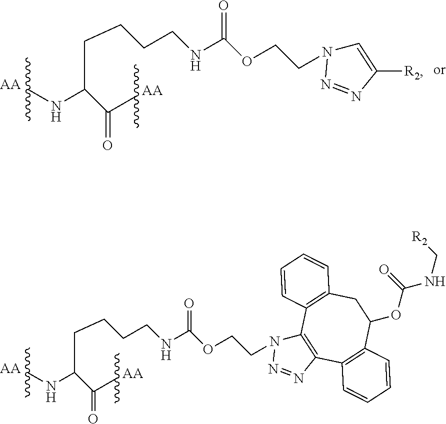

wherein,
the N is a mutation site, AA is an amino acid located before and after the mutation site, and
R₂ is a saccharide, a polynucleotide, an amino acid, a polypeptide or a carboxyl terminal modifying group.

In one embodiment of the present invention, the conjugate is a glycoconjugate formed by conjugating the carrier protein with site-directed mutation of the present invention to a polysaccharide containing or modified to have an alkynyl terminal group.

Preferably, the protein with site-directed mutation of the present invention is conjugated to a polysaccharide, wherein the ratio of the polysaccharide to the carrier protein (w/w) is 0.3 to 3.

Preferably, at least one covalent bond is present between the polysaccharide and the carrier protein every 10 to 50 saccharide repeat units of the polysaccharide.

The polysaccharide of the present invention can be a modified substance obtained by modification with an alkynyl terminal group, which realizes site-directed coupling by the catalysis of monovalent copper catalyst to obtain a glycoconjugate. Alternatively, the polysaccharide is a modified substance with cyclooctyne or a derivative thereof as a modifying group, which directly realizes site-directed coupling to a target polysaccharide protein.

The present application also provides an immunogenic composition comprising the glycoconjugate of the present invention and a pharmaceutically acceptable excipient, carrier or diluent.

The polysaccharide is selected from capsular polysaccharide; for example, the capsular polysaccharide is one of or a combination of two or more of pneumococcus serotype 1, 2, 3, 4, 5, 6A, 6B, 7F, 8, 9N, 9V, 10A, 11A, 12F, 14, 15B, 17F, 18C, 19A, 19F, 20, 22F, 23F and 33E

The present invention also provides a multivalent conjugate vaccine for pneumonia formed by multivalent pneumococcal polysaccharide and two or more carrier proteins with site-directed mutation of the present invention.

The present invention also provides use of the carrier protein with site-directed mutation, the conjugate of the carrier protein with site-directed mutation or the immunogenic composition in preparation of a vaccine.

Preferably, the vaccine is an influenza vaccine or a pneumonia vaccine.

Compared with other methods, the advantages of the present invention may be embodied in one or more of the following:
1. An unnatural amino acid can be introduced at any site of a protein, thereby creating a carrier protein that can be specifically modified at this site only;
2. Efficient and specific modification can be realized by utilizing the specific active group on the unnatural amino acid;
3. The modification of a specific site can weaken the activity of protease with specific functions, and thus reduce side effects and realize the effect of the carrier protein;
4. By optimizing modification conditions and utilizing cyclooctyne-mediated copper-free Click reaction, a modification reaction that is high in efficiency, harmless to proteins and simple and feasible can be realized.

### BRIEF DESCRIPTION OF THE DRAWINGS

FIG. 1 shows SDS-PAGE electrophoretograms of the mutated protein Hin47-HID and the polysaccharide-protein conjugates obtained by Click reaction;

FIG. 2a shows a particle size distribution plot of the polysaccharide-protein conjugate obtained by Click reaction;

FIG. 2b shows a particle size distribution plot of the polysaccharide-protein conjugate obtained by conventional coupling method; and

FIG. 3 shows ELISA results of type 3 conjugates obtained by different coupling modes after triple immune of rabbits.

### DETAILED DESCRIPTION

The present invention will be further illustrated with reference to the following specific examples. It should be understood that these examples are merely intended to illustrate the present invention rather than limit the scope of the present invention.

### Example 1

### Construction of Expression Plasmid of Carrier Protein with Site-directed Mutation

### 1: Selection of Mutation Sites

The three-dimensional structures of HID and HIN47 were predicted separately using online software phyre2.

10 key amino acids were selected on each of fusion proteins HIN47-HID and HID-HIN47. After the key amino acid sites were substituted, the fusion proteins can be bound to the polysaccharide at fixed points, and the residual protease activity of HIN47 is expected to be further reduced as well, which is beneficial to improve the stability of a final polysaccharide-binding product. The information of specific mutation sites is shown in Table 1 and Table 2, wherein an amino acid position refers to a position on sequences set forth in SEQ ID NOs: 2-3.

TABLE 1
HIN47-HID mutation sites

| Amino acid | Amino | Codon before | Codon after |
|---|---|---|---|
| position | acid | mutation | mutation |
| 522 | K | AAG | TAG |
| 557 | D | GAC | |
| 653 | E | GAA | |
| 684 | Y | TAT | |
| 174 | R | CGC | |
| 217 | I | ATC | |
| 223 | N | AAT | |
| 226 | I | ATT | |
| 313 | R | CGC | |
| 315 | K | AAA | |

TABLE 2
HID-HIN47 mutation sites

| Amino acid | Amino | Codon before | Codon after |
|---|---|---|---|
| position | acid | mutation | mutation |
| 78 | K | AAG | TAG |
| 113 | D | GAC | |
| 209 | E | GAA | |
| 240 | Y | TAT | |
| 527 | R | CGC | |
| 570 | I | ATC | |
| 576 | N | AAT | |
| 579 | I | ATT | |
| 666 | R | CGC | |
| 668 | K | AAA | |

### 2: Obtaining of Expression Plasmids

According to HID and HIN47 gene sequences published by NCBI GeneBank, full-length DNA fragments of the genes were each obtained through whole-gene synthesis. Then HIN47 and HID proteins were each fused and constructed, by using a GGSGGS six-amino-acid linker, in a pET9a vector in sequences of HIN47-linker-HID and HID-linker-HIN47, respectively, so that expression plasmids pET9a-HIN47-HID and pET9a-HID-HIN47 were obtained, respectively.

### 3: Site-Directed Mutation

The Fast Mutagenesis System site-directed mutation kit from TransGene Biotech was used, and according to the instruction of the kit, the expression plasmids pET9a-HIN47-HID and pET9a-HID-HIN47 were used as templates, and the mutation primers in Tables 3-4 were used for mutations. For the plasmids obtained after the mutation, sequencing verification was performed. Sequencing results showed that all genes at mutation sites were successfully mutated into TAG, and 10 plasmids with site-directed mutation were obtained.

The 10 mutant clones of the fusion protein pET9a-HIN47-HID were named: pET9a-HIN47-HID-R174, pET9a-HIN47-HID-I217, pET9a-HIN47-HID-N223, pET9a-HIN47-HID-I226, pET9a-HIN47-HID-R313, pET9a-HIN47-HID-K315, pET9a-HIN47-HID-K522, pET9a-HIN47-HID-D557, pET9a-HIN47-HID-E653, and pET9a-HIN47-HID-Y684.

The 10 mutant clones of the fusion protein pET9a-HID-HIN47 were named: pET9a-HID-HIN47-K78, pET9a-HID-HIN47-D113, pET9a-HID-HIN47-E209, pET9a-HID-HIN47-Y240, pET9a-HID-HIN47-R527, pET9a-HID-HIN47-1570, pET9a-HID-HIN47-N576, pET9a-HID-HIN47-I579, pET9a-HID-HIN47-R666, and pET9a-HID-HIN47-K668.

TABLE 3
List of mutation primers for HIN47-HID

| Mutation | Primer | Sequence | |
|---|---|---|---|
| site | coding | number | Primer sequence 5′ to 3′ |
| K522 | 522F | SEQ ID | CCGATGTAGCCtagAAATTTCCTCATAGGCATAGGAAAG |
| | | NO: 4 | |
| | 522R | SEQ ID | TTTctaGGCTACATCGGTTAAGCCGTCCAGAA |
| | | NO: 5 | |
| D557 | 557F | SEQ ID | GACCAAAtagGGGAAACAGGCTCAAGTGTATCC |
| | | NO: 6 | |
| | 557R | SEQ ID | GTTTCCCctaTTTGGTCTCGAAATTTTCCGTC |
| | | NO: 7 | |
| E653 | 653F | SEQ ID | AAAACCtagCTCCTTCCTCAGATGGGAATGGA |
| | | NO: 8 | |
| | 653R | SEQ ID | GGAAGGAGctaGGTTTTTATGCGTTTCAGTTCATT |
| | | NO: 9 | |
| Y684 | 684F | SEQ ID | CGAAAGGAtagTGGGTAAACTACAATTATGATTGGATG |
| | | NO: 10 | |
| | 684R | SEQ ID | TACCCActaTCCTTTCGGATCTTTTTCCTGGG |
| | | NO: 11 | |
| R174 | 174F | SEQ ID | tagAGTACCGGGTCAGACTCCGGCACCTACGA |
| | | NO: 12 | |
| | 174R | SEQ ID | TCTGACCCGGTACTctaGCCCAGAGCGGAAACTATCC |
| | | NO: 13 | |
| I217 | 217F | SEQ ID | TGCTATCtagTCACCGAGCGGCGGTAATGCGG |
| | | NO: 14 | |
| | 217R | SEQ ID | TCGGTGActaGATAGCAGTATTGATACCGATCAATTC |
| | | NO: 15 | |
| N223 | 223F | SEQ ID | TtagGCGGGAATTGCGTTCGCAATTCCCAGTA |
| | | NO: 16 | |
| | 223R | SEQ ID | ACGCAATTCCCGCctaACCGCCGCTCGGTGAGAT |
| | | NO: 17 | |
| I226 | 226F | SEQ ID | AATGCGGGAtagGCGTTCGCAATTCCCAGTAA |
| | | NO: 18 | |
| | 226R | SEQ ID | AACGCctaTCCCGCATTACCGCCGCTCGGTGA |
| | | NO: 19 | |
| R313 | 313F | SEQ ID | GCTGAAATTtagGCAAAAATTGCTACAACTGGTGC |
| | | NO: 20 | |
| | 313R | SEQ ID | TTTGCctaAATTTCAGCAAACGAAGAGATTTTC |
| | | NO: 21 | |
| K315 | 315F | SEQ ID | TCGCGCAtagATTGCTACAACTGGTGCAGGCA |
| | | NO: 22 | |
| | 315R | SEQ ID | TAGCAATctaTGCGCGAATTTCAGCAAACGAA |
| | | NO: 23 | |

TABLE 4
List of mutation primers for HID-HIN47

| Mutation | Primer | Sequence | |
|---|---|---|---|
| site | coding | number | Primer sequence 5′ to 3′ |
| K78 | 78F | SEQ ID | CCGATGTAGCCtagAAATTTCCTCATAGGCATAGGAAAG |
| | | NO: 24 | |
| | 78R | SEQ ID | TTTctaGGCTACATCGGTTAAGCCGTCCAGAA |
| | | NO: 25 | |
| D113 | 113F | SEQ ID | GACCAAAtagGGGAAACAGGCTCAAGTGTATCC |
| | | NO: 26 | |
| | 113R | SEQ ID | GTTTCCCctaTTTGGTCTCGAAATTTTCCGTC |
| | | NO: 27 | |
| E209 | 209F | SEQ ID | AAAACCtagCTCCTTCCTCAGATGGGAATGGA |
| | | NO: 28 | |
| | 209R | SEQ ID | GGAAGGAGctaGGTTTTTATGCGTTTCAGTTCATT |
| | | NO: 29 | |
| Y240 | 240F | SEQ ID | CGAAAGGAtagTGGGTAAACTACAATTATGATTGGATG |
| | | NO: 30 | |
| | 240R | SEQ ID | TACCCActaTCCTTTCGGATCTTTTTCCTGGG |
| | | NO: 31 | |
| R527 | 527F | SEQ ID | tagAGTACCGGGTCAGACTCCGGCACCTACGA |
| | | NO: 32 | |
| | 527R | SEQ ID | TCTGACCCGGTACTctaGCCCAGAGCGGAAACTATCC |
| | | NO: 33 | |
| I570 | 570F | SEQ ID | TGCTATCtagTCACCGAGCGGCGGTAATGCGG |
| | | NO: 34 | |
| | 570R | SEQ ID | TCGGTGActaGATAGCAGTATTGATACCGATCAATTC |
| | | NO: 35 | |
| N576 | 576F | SEQ ID | TtagGCGGGAATTGCGTTCGCAATTCCCAGTA |
| | | NO: 36 | |
| | 576R | SEQ ID | ACGCAATTCCCGCctaACCGCCGCTCGGTGAGAT |
| | | NO: 37 | |
| I579 | 579F | SEQ ID | AATGCGGGAtagGCGTTCGCAATTCCCAGTAA |
| | | NO: 38 | |
| | 579R | SEQ ID | AACGCctaTCCCGCATTACCGCCGCTCGGTGA |
| | | NO: 39 | |
| R666 | 666F | SEQ ID | GCTGAAATTtagGCAAAAATTGCTACAACTGGTGC |
| | | NO: 40 | |
| | 666R | SEQ ID | TTTGCctaAATTTCAGCAAACGAAGAGATTTTC |
| | | NO: 41 | |
| K668 | 668F | SEQ ID | TCGCGCAtagATTGCTACAACTGGTGCAGGCA |
| | | NO: 42 | |
| | 668R | SEQ ID | TAGCAATctaTGCGCGAATTTCAGCAAACGAA |
| | | NO: 43 | |

### Example 2

### Expression and Purification of Mutant Carrier Protein

### Lys-Azido Incorporation Expression and Purification of Mutant Proteins

The expression plasmid pET9a-HIN47-HID-K522 obtained in Example 1 was cultured in LB medium at 37° C. for 12 to 16 h, and then secondary amplification was carried out until the OD value of the bacterial solution was 0.6 to 1.0. Lys-azido was added until a final concentration of 1 mM was reached, and amplification was continued at 37° C. for 30 mM, and IPTG was added until a final concentration of 0.5 mM was reached. The final concentration of arabinose was 0.2%. Expression was induced at 24° C. for 12 h, and then bacteria were collected.

The collected bacteria were equilibrated and resuspended using Ni-NTA-Bind-Buffer, disrupted by ultrasonic, centrifuged to remove cell debris, purified by Ni-NTA metal chelating affinity chromatography, fully washed with Ni-NTA-Wash-Buffer and finally eluted with Ni-NTA-Elute-Buffer to obtain a preliminary purified protein sample HIN47-HID-K522 with a purity of about 90% (see FIG. 1, line 4).

### Example 3

### Site-Directed Coupling of Mutant Carrier Protein to Polysaccharide by Copper-Free Catalysis

The copper-free Click reaction needed to rely on the ring tension effect of cyclooctyne, and a modified substance was coupled to DIBO (polysaccharide with a cyclooctyne structure), so that the copper-free catalytic Click reaction with a group containing azido was carried out. The Click reaction system was as follows:

1 μg/μL HIN47-HID-K522 protein and DIBO-pneumonia type 3 polysaccharide (a molecular weight of 200 KD and 400 KD, 2 mM each) were vertically suspended at 4° C. for 2 hours. The results showed that pneumonia type 3 polysaccharide with different molecular weights (200 KD and 400 KD) could be successfully conjugated to the protein by modification to obtain F3-HIN47-HID-K522-200 and F3-HIN47-HID-K522-400 (see FIG. 1, lines 1 and 2).

By the reaction conditions described above, about 90% of proteins could be site-directly coupled with polysaccharides within 1 h. The re-dissolved substance after the reaction was desalted and then subjected to ion exchange, so that a protein with site-directedly coupled polysaccharide that had a purity >95% could be obtained.

### Example 4

### Coupling to Polysaccharide by Copper-Catalyzed Click Reaction

The reaction system was as follows:

| | | |
|---|---|---|
| HIN47-HID-K522 protein | 1 | μg/μL |
| Pneumonia type 1 polysaccharide-alkyne | 20 | μg/μL |
| Cu²⁺ | 1 | mM |
| BTTES | 400 | μmol |

| | |
|---|---|
| PBS | 0.01M (pH ≈ 7) |
| Cu wire segment | (sufficient amount) |

Note:
(BTTES was short for (1,2,3-triazol-1-yl) ethanesulfonic acid)

The reaction conditions were as follows: the reaction system was vertically suspended for 30 min at 4 ° C., and after the reaction was finished, EDTA was added until a concentration of 1 mM was reached to stop the reaction. The resulting final product was a conjugate F3-HIN47-HID-K522-Cu that was formed by site-directly coupling the pneumonia type 3 polysaccharide to the HIN47-HID-K522 protein (see FIG. 1, line 3), wherein the coupling of polysaccharide to the target protein is carried out through a copper-catalyzed Click reaction, as can be seen in FIG. 1.

### Example 5

### Evaluation of Filterability of Protein with Site-Directedly Coupled Polysaccharide

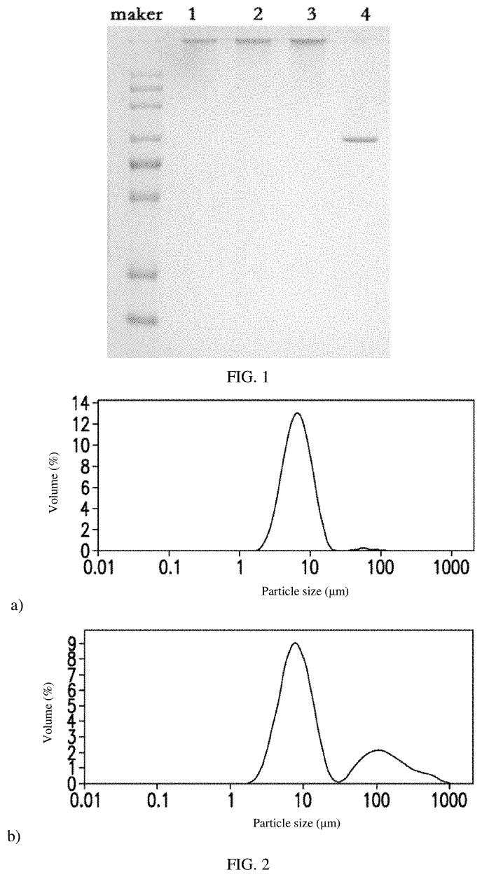

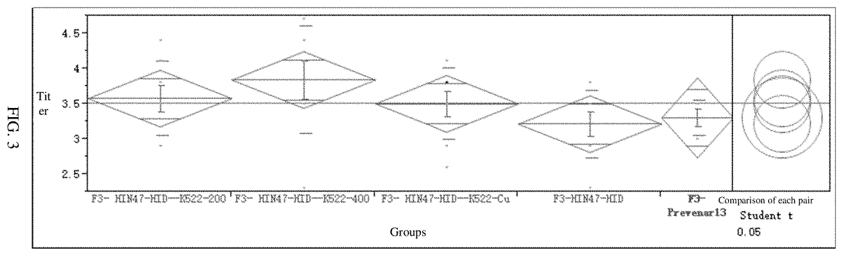

According to analysis of the particle size distribution of the conjugates using the Malvern laser particle size analyzer Mastersizer 2000, the particle size distribution of the conjugate formulation obtained by site-directly coupling polysaccharide to protein was uniform and free of obvious aggregates, but the conjugate F3-HIN47-HID-K522-Cu obtained by the conventional polysaccharide-protein coupling method was not uniform and contained aggregates (see FIG. 2b). The conventional polysaccharide-protein coupling method was as follows: type 3 polysaccharide was hydrolyzed at high temperature of 60° C. and then subjected to activation reaction in the presence of 0.5 g/L sodium periodate to obtain an activated polysaccharide; the activated polysaccharide mixed with carrier protein HIN47-HID in 0.1 M phosphate buffer solution (pH 6.5), sodium cyanoborohydride was added until a final concentration of 2 g/L was reached, and the reaction mixture reacted for 56 h; then 2 g/L sodium borohydride, was added, and the reaction was continued for two hours; and then ultrafiltration with normal saline was performed to obtain a conjugate F3-HIN47-HID.

### Example 6

### Evaluation on Immunogenicity of Protein Conjugate with Site-Directedly Coupled Polysaccharide

In the rabbit models, vaccines obtained by different coupling modes were studied. The study related to the effects of the polysaccharide-protein conjugates of the type 3 capsular polysaccharide from Streptococcus pneumoniae and the HIN47-HID proteins in Examples 3, 4 and 5 and aluminium phosphate (A1PO₄) adjuvant on the immune response of a monovalent vaccine in rabbits. These effects were characterized by serum IgG concentrations determined by antigen-specific ELISA and antibody functions determined by opsonophagocytic assay (OPA). FIG. 3 shows ELISA results of type 3 conjugates obtained by different coupling modes after triple immune of rabbits. The results show that polysaccharide-protein conjugates of the type 3 capsular polysaccharide from Streptococcus pneumoniae and H1N47-HID proteins in Examples 3, 4, and 5 showed significant antibody titers compared to the control.

At weeks 0, 2, and 4, New Zealand white rabbits were immunized intramuscularly with one-quarter dose (0.5 μg of polysaccharide) of the planned clinical dose (2 μg of polysaccharide) for human. Serums were collected at different time points. Serum-specific IgG was determined by ELISA and functional activity was assessed by bactericidal ability (OPA).

TABLE 5
Detection results of type 3 polysaccharide-protein conjugates

| | | Free | |
|---|---|---|---|
| | | polysac- | Free |
| Polysaccharide-protein | Polysaccharide-protein | charide | protein |
| conjugate | conjugation ratio | (%) | (%) |
| F3-HIN47-HID-K522-200 | 0.62 | 18 | 1.2 |
| F3-HIN47-HID-K522-400 | 0.98 | 12 | 1 |
| F3-HIN47-HID-K522-Cu | 0.85 | 16 | 1.6 |
| F3-HIN47-HID | 0.89 | 23 | 2.8 |

TABLE 6
Bactericidal ability (OPA) results of type
3 polysaccharide-protein conjugates

| Polysaccharide-protein conjugate | OPA50%) |
|---|---|
| F3- HIN47-HID--K522-200 | 698 |
| F3- HIN47-HID--K522-400 | 1098 |
| F3- HIN47-HID--K522-Cu | 773 |
| F3-HIN47-HID | 305 |
| F3-prevenar | 325 |

Table 5 shows the detection results of the type 3 polysaccharide-protein conjugates, and the results show that the type 3 polysaccharide-protein conjugates obtained by site-directed coupling have percentages of free polysaccharide and free protein that are significantly less than those of the unmutated protein carrier. Table 6 shows the bactericidal ability (OPA) results of the type 3 polysaccharide-protein conjugates, and the results show that the type 3 polysaccharide-protein conjugates obtained by site-directed coupling induce significantly higher antibody responses and specific bactericidal ability in rabbits.

### Example 7

### Study on Safety of Protein Conjugate with Site-Directedly Coupled Polysaccharide

3 polysaccharide-protein conjugates prepared in Examples 3 and 4 were sampled. 5 mice weighed 18-22 g and 2 guinea pigs weighed 250-350 g were selected for each sample, wherein each mouse was subcutaneously injected with 0.5 mL of sample at ventral area and each guinea pig was subcutaneously injected with 5.0 mL of sample at ventral area. The animals were observed for 7 days, and the body weight changes thereof were recorded. The results show that all the mice and the guinea pigs were healthy and had no abnormal reaction in the observation period, and the body weight of each mouse and guinea pig was increased at the end of observation period compared to that before injection. This shows that the site-directed coupling conjugate vaccines prepared by the present invention haven't been contaminated by exogenous toxic substances and they are free of unexpected unsafe factors.

Examples 2-7 take HIN47-HID-K522 as an example to demonstrate effect experiments of preparation, characterization, detection methods, immunogenicity evaluation and the like of the mutant carrier protein. Other carrier proteins with site-directed mutation of HIN47-linker-HID and HID-linker-HIN47 are prepared by the methods above, and after immunogenicity and safety evaluation, they can reach a level equivalent to HIN47-HID-K522. The results show that the type 3 polysaccharide-protein conjugates obtained by site-directed coupling induce significantly higher antibody responses and specific bactericidal ability in rabbits.

Although the present invention has been described with the preferred embodiments above, they are not intended to limit the scope of the claims. Since many possible variations and modifications can be made by one skilled in the art without departing from the conception of the present invention, the scope of the present invention is to be determined by the claims.

1. A carrier protein with site-directed mutation, wherein the carrier protein is selected from fusion proteins formed by one, two or more of diphtheria toxoid, a non-toxic mutant of diphtheria toxin, a bacterial outer membrane protein and a bacterially expressed protein, wherein an amino acid at at least one site on the carrier protein is mutated into an unnatural amino acid, and the unnatural amino acid contains an azido or alkynyl terminal group.

2. The protein with site-directed mutation of claim 1, wherein the carrier protein is selected from fusion proteins formed by one, two or more of diphtheria toxoid, non-toxic mutant of diphtheria toxin CRM197, group B meningococcal outer membrane protein, recombinant Pseudomonas aeruginosa exotoxin A, Haemophilusinfluenzae lipoprotein HID and Haemophilusinfluenzae heat shock protein HIN47; preferably, the carrier protein is selected from non-toxic mutant CRM197 of diphtheria toxin, Haemophilusinfluenzae lipoprotein HID, fusion protein HIN47-HID and fusion protein HID-HIN47.

3. The protein with site-directed mutation of claim 1, wherein the unnatural amino acid is a phenylalanine derivative, a tyrosine derivative, a glutamine derivative, an alanine derivative, a cysteine derivative, a serine derivative or a lysine derivative.

4. The protein with site-directed mutation of claim 1, wherein the unnatural amino acid is

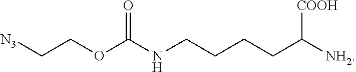 [SMILES: [N-]=[N+]=NCCOC(=O)NCCCCC(N)C(=O)O]

5. The protein with site-directed mutation of claim 1, wherein a mutation site of HIN47-HID fusion protein may be one or more amino acids at any site in SEQ ID NO: 2;
preferably, the mutation site is selected from position 174R, position 2171, position 223N, position 2261, position 313R, position 315K, position 522K, position 557D, position 653E and position 684Y on a sequence set forth in SEQ ID NO: 2.

6. The protein with site-directed mutation of claim 1, wherein a mutation site of HID-HIN47 fusion protein may be one or more amino acids at any site in SEQ ID NO: 3;
preferably, the mutation site is selected from position 78K, position 113D, position 209E, position 240Y, position 527R, position 5701, position 576N, position 5791, position 666R and position 668K on a sequence set forth in SEQ ID NO: 3.

7. The protein with site-directed mutation of claim 1, wherein an amino acid at position N on an amino acid sequence of a protein before mutation is mutated into Lys-azido, and a linking mode of the mutated amino acid is shown as the following formula: 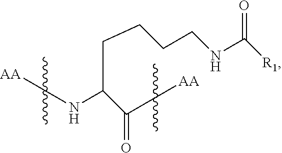
wherein, the N is a mutation site, and AA is an amino acid located before and after the mutation site, and R₁ is

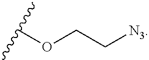 [SMILES: *OCCN=[N+]=[N-]]

8. A conjugate of a carrier protein with site-directed mutation, wherein the conjugate is prepared by the carrier protein with site-directed mutation of claim 1 and a saccharide, a polynucleotide, an amino acid, a polypeptide or a small molecule compound containing or modified to have an alkynyl terminal group.

9. The conjugate of the carrier protein with site-directed mutation of claim 8, wherein the amino acid at position N on the amino acid sequence of the protein before mutation is mutated into the following structure: 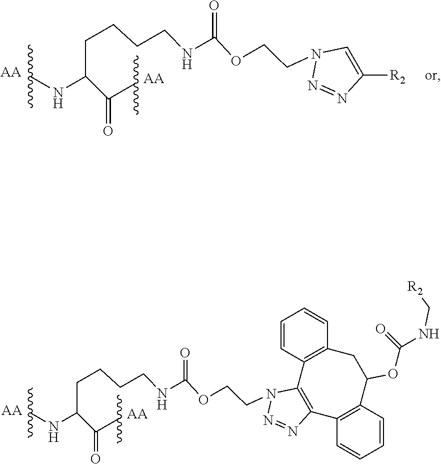
wherein,
the N is a mutation site, AA is an amino acid located before and after the mutation site, and
R₂ is a saccharide, a polynucleotide, an amino acid, a polypeptide or a carboxyl terminal modifying group.

10. The conjugate of the carrier protein with site-directed mutation of claim 8, wherein the conjugate is a glycoconjugate formed by conjugating the carrier protein with site-directed mutation to a polysaccharide containing or modified to have an alkynyl terminal group.

11. The conjugate of the carrier protein with site-directed mutation of claim 10, wherein the ratio of the saccharide to the carrier protein (w/w) in the glycoconjugate is 0.3 to 3.

12. The conjugate of the carrier protein with site-directed mutation of claim 10, wherein at least one covalent bond is present between the polysaccharide and the carrier protein every 10 to 50 saccharide repeat units of the polysaccharide.

13. An immunogenic composition comprising the glycoconjugate of claim 11, and a pharmaceutically acceptable excipient, carrier or diluent.

14. The immunogenic composition of claim 13, wherein the glycoconjugate is a glycoconjugate of a capsular polysaccharide selected from one of or a combination of two or more of: pneumococcus serotype 1, 2, 3, 4, 5, 6A, 6B, 7F, 8, 9N, 9V, 10A, 11A, 12F, 14, 15B, 17F, 18C, 19A, 19F, 20, 22F, 23F and 33F.

15. (canceled)

16. (canceled)

17. (canceled)
